(19) United States
(12) Patent Application Publication
(10) Pub. No.: US 2025/0284736 A1
(43) Pub. Date: Sep. 11, 2025

(54) METHOD, SYSTEM, AND MEDIUM FOR MEASURING, CALIBRATING AND TRAINING PSYCHOLOGICAL ABSORPTION

(71) Applicant: Lucid Inc., Toronto (CA)

(72) Inventors: Aaron Labbé, Toronto (CA); Frank Russo, Toronto (CA)

(21) Appl. No.: 18/853,100

(22) PCT Filed: Mar. 31, 2023
(86) PCT No.: PCT/CA2023/050440

§ 371 Date: Sep. 30, 2024

Related U.S. Application Data
(60) Provisional application No. 63/326,472, filed on Apr. 1, 2022.

(51) Int. Cl. G06F 16/638; A61B 3/11; A61B 3/113; A61B 5/00; A61B 5/0205; A61B 5/024; A61B 5/08; A61B 5/31; A61B 5/374; G10H 1/00

(52) U.S. Cl. CPC G06F 16/638; A61B 3/112; A61B 3/113; A61B 5/0075; A61B 5/0205; A61B 5/31; A61B 5/374; A61B 5/7267; G10H 1/0008; A61B 5/02416; A61B 5/0816; G10H 2210/031; G10H 2240/135; G10H 2240/141

(57) ABSTRACT

A method, system, and medium for measuring and training psychological absorption using music. A state absorption measure of a human subject is computed by obtaining biometric data from the human subject, and processing the biometric data to compute the state absorption measure. A personalized music library may be generated based on musical state absorption. A human subject may be trained to develop increased trait absorption using music.

### FIELD

At least some example embodiments relate to systems for psychological measurement, and in particular to systems for measuring and training psychological absorption using music.

### BACKGROUND

The psychological state of “absorption” can be defined as a period of almost ‘total’ immersion that consumes all of one's attentional and representational resources (perceptual, enactive, imaginative, and ideational). [See Tellegen A, Atkinson G. Openness to absorbing and self-altering experiences. Journal of Abnormal Psychology. 1974; 83 (3): 268-77. PMID: 4844914.] Moments of high absorption can result in strong nostalgic imprints in memory known as “flashbulb memory”, created after exceptional events that endure for long periods of time, retaining many details that would be easily forgotten in “ordinary” everyday memories. The state of absorption is closely linked to self-transcendence, awe, and spiritual experience. [See Lifshitz, M., van Elk, M., & Luhrmann, T. M. (2019). Absorption and spiritual experience: A review of evidence and potential mechanisms. Consciousness and cognition, 73, 102760.]

Absorption has also been considered as a trait variable, wherein some individuals are more likely to become absorbed by stimuli than others. Trait absorption is closely linked to the psychological constructs of imaginative involvement and openness to experience. [See Roche, S. M., & McConkey, K. M. (1990). Absorption: Nature, assessment, and correlates. Journal of personality and social psychology, 59 (1), 91.]

Absorption for music has been considered as both a state and trait variable.

Musical state absorption is strongly correlated with music preference but is independent of the music's emotional valence (i.e., whether the music conveys a positive or negative mood). [See Hall, S. E., Schubert, E., & Wilson, S. J. (2016). The role of trait and state absorption in the enjoyment of music. PloS one, 11 (11), e0164029.]

State absorption can be measured through simple self-report questions [See Hall; also Lange, E. B., Zweck, F., & Sinn, P. (2017). Microsaccade-rate indicates absorption by music listening. Consciousness and cognition, 55, 59-78] or through physiological responses [See Van Elk, M., Arciniegas Gomez, M. A., van der Zwaag, W., Van Schie, H. T., & Sauter, D. (2019). The neural correlates of the awe experience: Reduced default mode network activity during feelings of awe. Human brain mapping, 40 (12), 3561-3574; also Hu, X., Yu, J., Song, M., Yu, C., Wang, F., Sun, P., . . . & Zhang, D. (2017). EEG correlates of ten positive emotions. Frontiers in human neuroscience, 11, 26]. Studies involving neuroimaging indicate that state absorption leads to a unique pattern of brain activations resembling psychedelic states that are distinct from that which would be expected under analytical observation of the same stimuli [See Van Elk]. In particular, state absorption is accompanied by reduced activation in the default mode network (DMN; suggesting less self-referential thinking). Other features may include increased activation of the frontoparietal attention network (suggesting increased attention) and the human mirror neuron system (hMNS; suggesting empathy). Neuroelectric correlates of these network changes have also been documented and are characterized by increased power in high-frequency oscillatory activity (theta band) and decreased power in high-frequency oscillatory activity (alpha, beta). These findings suggest that it should be possible to use hemodynamic or neuroelectric measurement to assess state absorption as well as changes in trait absorption that may be realized through training.

Musical trait absorption has been defined as the capacity of music to induce and regulate emotions [See Sandstrom, G. M., & Russo, F. A. (2013). Absorption in music: Development of a scale to identify individuals with strong emotional responses to music. Psychology of Music, 41 (2), 216-228], as well as the potential to use music to enter into states of self-transcendence (loss of self-consciousness, ‘awe’ emotions, mystical experiences, disorientations of time and space) [Cardona, G., Ferreri, L., Lorenzo-Seva, U., Russo, F. A., Rodiguez-Fornells, A. (under review). The forgotten role of absorption in music reward. Annals of the New York Academy of Sciences]. Musical trait absorption may be measured by psychometric scales [See Sandstrom] and through biomarkers.

Thus, it would be desirable to measure state absorption, for example to enable the generation of a personalized music library and/or to train a human subject to increase trait absorption using music.

### SUMMARY

The present disclosure describes example devices, methods, systems, and non-transitory media for measuring psychological absorption through biometric measures, music preference discovery using measured absorption levels and training psychological absorption using music.

According to some aspects, the present disclosure is directed to a method for computing a state absorption measure of a human subject, comprising obtaining biometric data from the human subject, and processing the biometric data to compute the state absorption measure. The biometric data includes one or more of the following: functional near infrared spectroscopy (fNIRS) data, electroencephalography (EEG) data, eye tracking data, and photoplethysmography (PPG) data.

According to some aspects, the present disclosure is directed to a method for generating a personalized music library. A plurality of music segments are presented to a human subject. A state absorption measure of the human subject is determined during the presentation of each music segment. A musical trait absorption measure is computed for each music segment based on the state absorption measure of the human subject during the presentation of each music segment. At least one music segment of the plurality of music segments is selected for inclusion in the personalized music library based on the musical trait absorption measure of the at least one musical segment with respect to the human subject. Through machine learning methods, similar music segments are then found on the basis of feature similarity to the included segments in the personalized music library, enabling a larger library of music that reflects the preferences of the human subject.

According to some aspects, the present disclosure is directed to a method for training a human subject to develop increased trait absorption. A music library is obtained comprising a plurality of music segments likely to induce high trait absorption in the human subject. The human subject is instructed to perform one or more interactive exercises. While the human subject is performing the interactive exercises, one or more of the plurality of music segments is presented to the human subject.

### BRIEF DESCRIPTION OF THE DRAWINGS

Embodiments will now be described by way of examples with reference to the accompanying drawings, in which like reference numerals may be used to indicate similar features.

FIG. 1 is a block diagram of an example system for affective music recommendation according to example embodiments described herein.

FIG. 2 is a schematic diagram of the operation of a biometric classifier according to example embodiments described herein.

FIG. 3 is a front upper view of the face of a human subject showing the skull location of fNIRS sensors for biometric determination of state absorption according to example embodiments described herein.

FIG. 4 is a schematic diagram showing identification of flashbulb memory events according to example embodiments described herein.

FIG. 5 is a schematic diagram showing a machine learning classification module for state absorption according to example embodiments described herein.

FIG. 6 is a schematic diagram showing music preference discovery through absorption calibration according to example embodiments described herein.

FIG. 7 is a schematic diagram showing music preference updating using music recommendation systems according to example embodiments described herein.

FIG. 8 is a schematic diagram showing generation of a personalized music library based on musical trait absorption according to example embodiments described herein.

FIG. 9 is a schematic diagram showing an absorption calibration process including a human subject and a user device according to example embodiments described herein.

FIG. 10 is a schematic diagram showing an absorption training process according to example embodiments described herein.

FIG. 11 is a schematic diagram showing a content-based music segment candidate generation process according to example embodiments described herein.

### DETAILED DESCRIPTION OF EXAMPLE EMBODIMENTS

Example embodiments will now be described with respect to methods, systems, and non-transitory media for measuring psychological absorption through biometric measures, music preference discovery using measured absorption levels and training psychological absorption using music.

In some examples, systems and methods are provided for measuring state absorption in real-time through biometric inputs. This provides a powerful means of objectively measuring candidate moments of awe and transcendence while human subjects engage in multi-sensory environments, which may have applications in the context of various therapeutic practices, most notably psychedelic-assisted therapy.

In some examples, systems and methods are provided for calibrating or generating music libraries based on a human subject's state absorption using psychometric and biometric measures. A framework is also provided for training trait absorption to enhance a human subject's ability to become absorbed by music. Both frameworks leverage learning algorithms and cutting-edge bio-sensing technology for execution. Through a state absorption calibration method, an intuitive framework is provided for determining music preference without the need for manual input from the human subject, which may have applications in the context of music entertainment services, music-based therapeutic practices, and reminiscence therapy (especially for non-communicative patients). In some examples, a trait absorption training module provides the means for inducing heightened states of transcendence through music, which may have applications in the context of various therapeutic practices, most notably psychedelic-assisted therapy and music therapy.

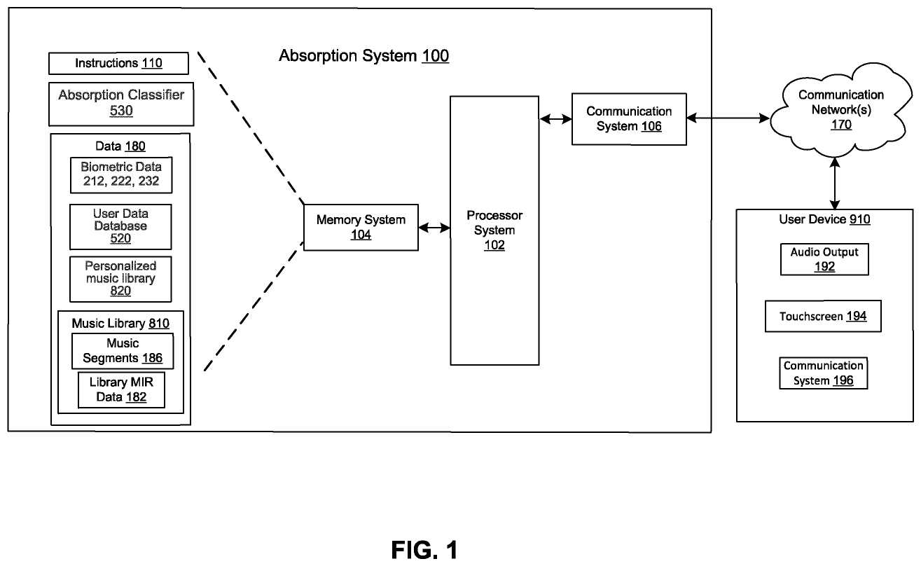

FIG. 1 shows an absorption system 100 including a processor system 102 for executing computer program instructions, a memory system 104 for storing executable instructions and data, and a communication system 106 for communicating data with other devices or components.

The absorption system 100 may be implemented on one or more computer systems. It may be embodied by a single computer, multiple computers, a virtual machine, a distributed computing or cloud computing platform, or any other platform of platforms capable of carrying out the method steps described herein. In some embodiments, the absorption system 100 may encompass one or more electronic devices used by human subjects (user devices 190), while in other embodiments the absorption system 100 is in communication with such devices, directly or indirectly (e.g. via a communication network 170) using the communication system 106.

The processor system 102 may be embodied as any processing resource capable of executing computer program instructions, such as one or more processors on a computer or computing platform(s). The memory system 104 may be embodied as any data storage resource, such as one or more disk drives, random access memory, or volatile or non-volatile memory on one or more computing platforms. The communication system 106 may be embodied as one or more communication links or interfaces, including wired or wireless communication interfaces such as Ethernet, Wifi, or Bluetooth interfaces. In some embodiments, one or more of the user devices 190 may be implemented on the same platform as the absorption system 100; in such embodiments, the communication system 106 may comprise an internal communication bus or other intra-platform data transfer system.

The memory system 104 may have stored thereon several types of computer programs in the form of executable instructions. There may be stored thereon a set of executable instructions 110 for carrying out the method steps described herein. There may also be one or more models (not shown), such as machine learning models, content filtering models, and/or collaborative filtering models, for performing functions described herein such as identifying audio segments intended to induce high state absorption in a listener, identifying music segments likely to align with a human subject's preferences, and/or personalizing an absorption training regimen. These models may be deployed on the absorption system 100 after being trained as further described below.

The memory system 104 may have stored thereon several types of data 180. The data 180 may include biometric data such as fNIRS data 212, EEG data 222, video data 232, and/or other biometric data as described below, in raw and/or preprocessed format(s). The data 180 may also include a music library 810, comprising a plurality of music segments 186 and music feature data corresponding to each of the plurality of music segments 186. The music segments 186 may comprise digital audio data stored as individual audio clips, or they may be extracts from audio clips stored in the music library 810, such as epochs of fixed duration extracted from songs of variable durations. The music feature data is shown here as library MIR data 182. It may include music information retrieval (MIR) metadata associated with each music segment 186 indicating MIR features of the music segment 186 with corresponding values. The music feature data may also, in some embodiments, include non-MIR data or metadata.

The user device 190 may be an electronic device operated by a human subject (e.g., an end user or 3rd party user like a therapist or caregiver) of the absorption system 100, such as a computer or smart phone in communication with the absorption system 100 via the communication network 170. The absorption system 100 may support multiple types of user device 190. Some user devices 190 include user interface components, such as a touchscreen 194 for displaying visual data and receiving user input and an audio output 192, such as speakers and/or a wired or wireless interface to headphones. Communication with the absorption system 100 is enabled via a communication system 196, which may communicate via the communication network 170.

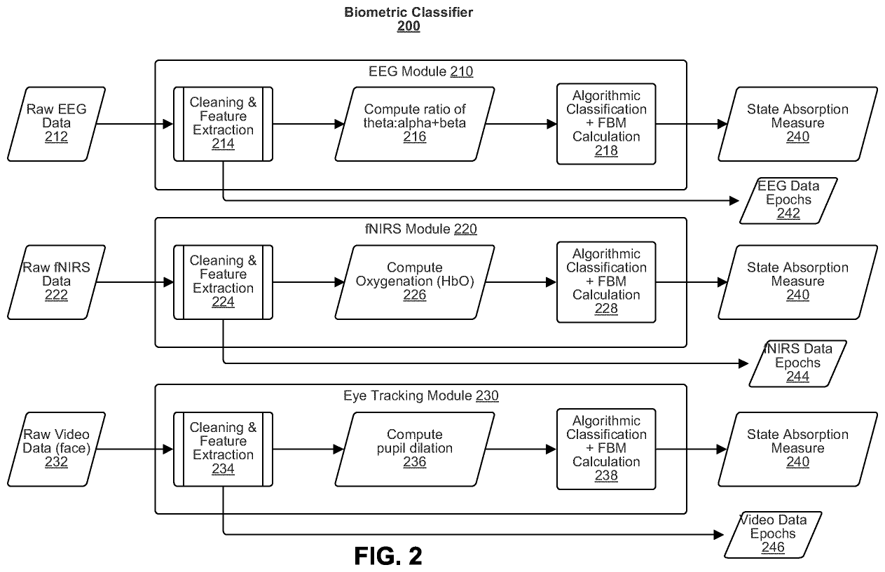

FIGS. 2-11 show various functional subsystems or modules of the absorption system 100 and/or the user device 190. Various functional steps are carried out by the absorption system 100 by using the processor system 102 to execute the executable instructions 110 stored in the memory system 104.

FIG. 2 shows an example biometric classifier 200 implemented by the absorption system 100 for performing biometric measurement of the state absorption of a human subject. Various biomarkers that may be collected outside of the laboratory reveal systemic changes indicative of state absorption, opening the door for ongoing biometric assessment of absorption in the real world. Promising tools for such measurement include hemodynamic changes via functional near infrared spectroscopy (fNIRS), neuroelectric changes via electroencephalography (EEG), pupil dilation, and rate of microsaccades via video-based eye tracking, and heart rate via photoplethysmography (PPG). By combining these metrics and leveraging machine learning, tracking efficacy may be optimized for a given individual human subject.

The biometric classifier 200 implements a process for building one or more software-based classifiers for absorption and/or flashbulb memory potential using various biometric devices. The software-based classifiers may include an EEG module 210, a fNIRS module 220, an eye tracking module 230, and/or one or more additional software-based classifiers for classifying or predicting state absorption based on biometric data. These software classifiers can either be implemented locally (for example, on user device 910) or in a cloud-based computing environment (for example, on absorption system 100).

In some embodiments, either or both of two options may be used for biometric classification of state absorption. The first is a formulaic algorithmic approach that cleans and converts raw biometric data into a measure that directly maps to state absorption. This option may be best used with the high probability metrics listed below, i.e. EEG, fNIRS, and eye tracking. The second leverages a machine learning process to build software classifiers by correlating ground truth psychometric data with various biometric features, potentially allowing for a more flexible system and opening the door for the use of lower probability peripheral measurements. The machine learning option also allows for personalized classifiers that can account for variance in an individual's physiology and trait absorption levels. In both options, a secondary formula can be used to calculate moments of flashbulb memory potential or “FBM potential”, which may indicate the occurrence of a flashbulb memory event (FBM event). In some examples, FBM potential is indicated by high absorption levels sustained for periods of at least 20 seconds long. These epoch lengths and the definition of a “high absorption” level can be defined depending on the embodiment. Both options may present advantages in measuring absorption relative to existing approaches, such as preventing the need for state absorption surveys and thereby effectively opening the door for time series absorption data.

Because of the limited amount of research existing on biometric measures of absorption, the brain-based and peripheral biometric measures described herein have been identified in consultation with literature on aligned experiences, such as “awe” mystical experiences, and psychedelics. The biometric measures used by the biometric classifier 200 may include high potential measures that are highly correlated with high sate absorption in the literature, and/or peripheral measures that may demonstrate a less straightforward relationship with high sate absorption in the literature.

The fNIRS module 220 may be used to process raw fNIRS data 222 to compute a state absorption measure 240 for a human subject. Using fNIRS, the fNIRS module 220 may track absorption through decreased activity in default mode network (DMN), and increased activity in the frontoparietal attention network and the human mirror neuron system (hMNS). This may be further quantified as a ratio of activity between the former and latter two networks (i.e., frontoparietal attention network and hMNS). Support for this correlation is described by [Van Elk, M., Arciniegas Gomez, M. A., van der Zwaag, W., Van Schie, H. T., & Sauter, D. (2019). The neural correlates of the awe experience: Reduced default mode network activity during feelings of awe. Human brain mapping, 40 (12), 3561-3574]. It will be appreciated that that Van Elk et al. (2019) suggest that the neural mechanisms associated with awe may also underpin other types of self-transcendental experiences such as peak moments in psilocybin effects that induce ego dissolution. See also [Carhart-Harris, R. L., Erritzoe, D., Williams, T., Stone, J. M., Reed, L. J., Colasanti, A., . . . & Nutt, D. J. (2012). Neural correlates of the psychedelic state as determined by fMRI studies with psilocybin. Proceedings of the National Academy of Sciences, 109 (6), 2138-2143].

The fNIRS module 220 initially processes the fNIRS data 222 using a cleaning and feature extraction process 224, which operates as follows, at a high level. First, channel pruning is performed to remove channels that have saturated or have not received sufficient light. This can be performed post data-collection through the use of an automated function in a matlab toolbox called Homer3. Second, intensity data can be converted into Optical Density (OD). Third, a low pass or band pass filter is applied. The cut-off for the low pass may be set to 0.1 Hz. The purpose of this filter is to suppress physiological noise and high frequency instrument noise. Optionally, if short channels exist, the data is submitted to a general linear model (GLM) using a low-pass filter with a cut-off at 5 Hz. This may suppress the high frequency instrument noise but maintain physiological noise (i.e. the heart beat). The physiological noise may be retained during processing by the GLM, because it acts as quality assurance; the presence of the heart beat indicates good optode-scalp coupling. Next, short channel physiological noise may be eliminated by performing a regression.

The data is then split into epochs, such as 10-second windows with 5-seconds overlap between windows in the channels of interest (i.e., the fNIRS measurements of activity in the human subject's medial prefrontal cortex).

The feature extraction process 224 then detects movements and applies movement correction. At this stage, if the data does not include a short channel, some embodiments may use methods that aim to remove systemic physiological noise that overlaps with the bandwidth of interest—e.g., using a low-pass filter to remove the heart rate signal. The epochs of fNIRS data 244, before or after movement correction and/or removal of systemic physiological noise, may be generated as an output of the fNIRS module 220 in some examples.

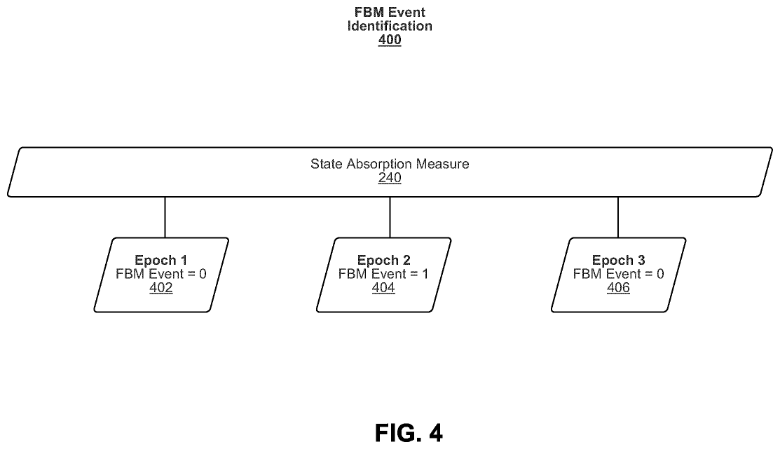

An oxygenation calculation process 226 of the feature extraction process 224 then converts optical density to oxygenation (HbO) and deoxygenation (HbR). An average is then calculated over the epochs to assess oxygenation levels in 10-second intervals. The oxygenation level may be used by an algorithmic classification and FBM calculation process 228 to compute the state absorption measure 240, which is generated as an output of the fNIRS module 220. The algorithmic classification and FBM calculation process 228 may also identify FBM events, as described below with reference to FIG. 4.

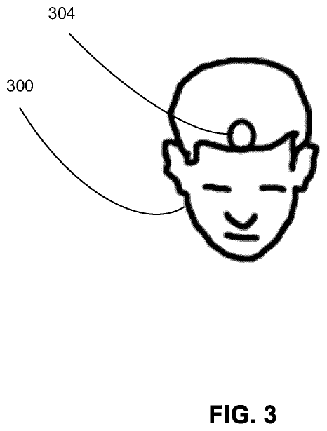

FIG. 3 is a front upper view of the face of a human subject 300 showing the skull location 304 of FNIR sensors for biometric determination of state absorption in some embodiments of the fNIRS module 220.

Open-source software code supporting fNIRS analyses may be used in some embodiments of the fNIRS module 220, such as the HomER software system described by [Huppert, T. J., Diamond, S. G., Franceschini, M. A., & Boas, D. A. (2009). HomER: a review of time-series analysis methods for near-infrared spectroscopy of the brain. Applied optics, 48 (10), D280-D298].

The EEG module 210 may be used to process raw EEG data 212 to compute a state absorption measure 240 for a human subject. Using EEG band power analysis, the EEG module 210 may track state absorption through increases in theta wave power and decreases in high-frequency band power (alpha waves, beta waves). This may be further quantified as a ratio, e.g., theta to (alpha+beta).

The relationship between EEG and state absorption is based at least in part on findings described in the literature. Increased theta-band activity may be associated with quieting of DMN as well as positive emotions like amusement, interest and joy: this finding implies that fronto-medial electrodes may be most relevant for collecting the EEG data 212. See [Scheeringa, R., Bastiaansen, M. C., Petersson, K. M., Oostenveld, R., Norris, D. G., & Hagoort, P. (2008). Frontal theta EEG activity correlates negatively with the default mode network in resting state. International jour-nal of psychophysiology, 67 (3), 242-251. See also Van Elk, M. (2014). An EEG study on the effects of induced spiritual experiences on somatosensory processing and sensory suppression. Journal for the Cognitive Science of Religion, 2 (2), 121]. See also [Hu, X., Yu, J., Song, M., Yu, C., Wang, F., Sun, P., . . . & Zhang, D. (2017). EEG correlates of ten positive emotions. Frontiers in human neuroscience, 11, 26].

Decreased alpha and beta-band activity is also observed under the influence of psychedelics and during emotional listening to song fragments. In particular, desynchronization (reduction in power) across all frequency bands alpha and above for frontal electrodes may indicate high state absorption. See [Muthukumaraswamy, S. D., Carhart-Harris, R. L., Moran, R. J., Brookes, M. J., Williams, T. M., Errtizoe, D., . . . & Nutt, D. J. (2013). Broadband cortical desynchronization underlies the human psychedelic state. Journal of Neu-roscience, 33 (38), 15171-15183]. See also [McGarry, L. M., Pineda, J. A., & Russo, F. A. (2015). The role of the extended MNS in emotional and nonemotional judgments of human song. Cognitive, Affective, & Behavioral Neuroscience, 15 (1), 32-44].

The EEG module 210 initially processes the EEG data 212 using a cleaning and feature extraction process 214, which operates as follows, at a high level. First, a band-pass filter is applied, having a high-pass cutoff at 1 Hz and a low-pass cutoff at 55 Hz. Powerline interference at 60 Hz is also suppressed-the band-pass filter may not be able to filter this out. Second, a reference value is subtracted from each electrode; the reference may be computed as an average over all electrode measurements, or to a mastoid electrode if available.

Third, the data is split into epochs, such as 10-second windows with 5-seconds overlap between windows. Fourth, bad data is removed, e.g. by applying the Clean_rawdata function in EEGLab (which performs automatic bad data rejection). The EEG data epochs 242, before or after bad data removal, may be provided as an output of the EEG module 210.

The ratio calculation module 216 then calculates a ratio of theta wave power to (alpha wave power+beta wave power) for each epoch. For each epoch, the ratio calculation module 216 computes a normalized power in each of the following frequency bands: theta (4-7 Hz), alpha (8-12 Hz), and beta (13-20 Hz). The process for normalization may be dividing power in the band of interest over the entire frequency range of interest (i.e., 4-20 Hz) rather than the entire frequency range available (i.e., 1-100 Hz). This calculation may help to indicate the relative amount of power driven by each band of interest.

The algorithmic classification and FBM calculation process 218 then calculates the state absorption measure 240, e.g., as equal to the ratio of theta wave power to (alpha wave power+beta wave power) for each epoch.

Open-source software code to support EEG spectrotemporal analyses may be used in some embodiments, such as the EEGLAB software described by [Delorme, A., & Makeig, S. (2004). EEGLAB: an open source toolbox for analysis of single-trial EEG dynamics including independent component analysis. Journal of neuroscience methods, 134 (1), 9-21].

The eye tracking module 230 may be used to process raw video data 232 showing the face of a human subject to compute a state absorption measure 240. Using video-based eye tracking, the eye tracking module 230 may track state absorption through pupil dilation and the rate of microsaccades.

Pupil dilation is a widely used measure of listening effort [see Winn, M. B., Edwards, J. R., & Litovsky, R. Y. (2015). The impact of auditory spectral resolution on listening effort revealed by pupil dilation. Ear and hearing, 36 (4), e153.], as well as autonomic arousal, and chills. Pupil dilation can be measured using a smartphone camera and video-based motion tracking software. See [Liao, H. I., Yoneya, M., Kashino, M., & Furukawa, S. (2018)]. A study of awe involving electromyography (EMG) data revealed a facial pattern resembling freezing. Thus, tracking movement in eye gaze and facial features may enable computation of state absorption. See [Chirico A., Cipresso P., Yaden D. B., Biassoni F., Riva G., Gaggioli A. (2017). Effectiveness of immersive videos in inducing awe: an experimental study. Sci. Rep. 7:1218. 10.1038/s41598-017]. Another study revealed zygomaticus activity consistent with smiling in subjects experiencing a transcendent state: see [Clayton R. B., Raney A. A., Oliver M. B., Neumann D., Janicke-Bowles S. H., Dale K. R. (2019). Feeling transcendent? Measuring psychophysiological responses to self-transcendent media content. Media Psychol. 1, 1-26]. Thus, it may be useful to analyze video data to detect a half-smile with minimal head motion—i.e., measure the extent of motion of the subject's face in addition to zygomaticus muscle activity.

The eye tracking module 230 initially processes the video data 232 using a cleaning and feature extraction process 234, which operates as follows, at a high level. First, video data 232 is obtained. In some embodiments, the video data 232 consists of a sequence of video frames, each including a full frontal image of the subject's face.

Second, the raw video data 232 is processed to identify at least one pupil of the subject in the video frames. In some embodiments, the video data 232 is subjected to PupilEXT, an open-source analysis framework for pupil image extraction.

Third, the pupil video data is split into epochs such as 10-second windows with 5-seconds overlap between windows. The video data epochs 246, before or after pupil extraction, may be generated as an output of the eye tracking module 230.

The pupil dilation and microsaccade computation process 236 processes the pupil data epochs to determine the degree of pupil dilation and the number of microsaccades in each epoch. Each microsaccade is determined using a thresholding method after [Engbert, R., & Mergenthaler, K. (2006). Microsaccades are triggered by low retinal image slip. Proceedings of the National Academy of Sciences, 103 (18), 7192-7197]. The algorithmic classification and FBM calculation process 218 then calculates the state absorption measure 240, e.g., corresponding to the degree of pupil dilation and rate of microsaccades.

In some embodiments, open-source software code may be used to support pupil dilation and microsaccade analysis, such as the PupilEXT software described by [Zandi, B., Lode, M., Herzog, A., Sakas, G., & Khanh, T. Q. (2021). PupilEXT: Flexible open-source platform for high-resolution pupillom-etry in vision research. Frontiers in neuroscience, 603]. The software and documentation may be obtained from:

[https://www.frontiersin.org/articles/10.3389/fnins.2021.676220/full], or [https://github.com/openPupil/Open-PupilEXT].

In some embodiments, head movement and/or zygomaticus activity may also be detected in the video data and used to compute the state absorption measure 240 as described above, using a further video-based software classifier (not shown). First, to assess extent of movement, the raw video data 232 may be subjected to an optical flow analysis, e.g., using FlowAnalyzer as described by [Barbosa, A. V., Yehia, H. C., & Vatikiotis-Bateson, E. (2008). Linguistically valid movement behavior measured non-invasively. In AVSP (pp. 173-177)]. The open source FlowAnalyzer software computes pixel displacements between consecutive frames in video. All pixel displacements are represented as vectors representing magnitude and 2D direction. Those vectors are then summed into a single vector representing magnitude of movement over time.

Second, the data is split into epochs such as 10-second windows with 5-seconds overlap between windows. The state absorption measure 240 may be generated based on epochs having low amounts of facial movement.

Smile detection may be used in some embodiments, as described above, using the raw video data 232. Open-source code to support video-based smile tracking is described by [Eyben, F., Weninger, F., Gross, F., & Schuller, B. (2013 October). Recent developments in opensmile, the munich open-source multimedia feature extractor. In Proceedings of the 21st ACM international conference on Multimedia (pp. 835-838)].

As described above, additional peripheral biometric or physiological measures may be used in some embodiments to compute the state absorption measure 240. It will be appreciated that each physiological measure can yield multiple features—e.g., photoplethysmography (PPG) data can be processed to generate respiration, heart rate, and heart-rate variability data. Heart-rate variability data can be further processed to generate repetition rate data (through autocorrelation) and/or high-frequency and/or low-frequency signal components. Peripheral biometric measures that may correlate with state absorption are discussed in an NIH publication at

[https://www.ncbi.nlm.nih.gov/pmc/articles/PMC7701337/#B4].

There are several physiological processes correlated to awe, transcendence and absorption experiences. Unlike brain measures, these physiological processes are best understood as consequences rather than causes of absorption. Galvanic skin response (GSR) data may be processed to generate a state absorption measure 240, based on findings suggested by [Clayton R. B., Raney A. A., Oliver M. B., Neumann D., Janicke-Bowles S. H., Dale K. R. (2019). Feeling transcendent? Measuring psycho-physiological responses to self-transcendent media content. Media Psychol. 1, 1-26. 10.1080/15213269.2019.1700135]. See also [Shiota M. N., Campos B., Oveis C., Hertenstein M. J., Simon-Thomas E., Keltner D. (2017). Beyond happiness: building a science of discrete positive emotions. Am. Psychol. 72:617. 10.1037/a0040456].

Respiration rate can be abstracted from PPG data. Slowed respiration rate may indicate high absorption. See [Ahani A., Wahbeh H., Nezamfar H., Miller M., Erdogmus D., Oken B. (2014). Quantitative change of EEG and respiration signals during mindfulness meditation. J. Neuroeng. Rehabil. 11, 1-11. 10.1186/1743-0003-11-87]. See also [Wielgosz J., Schuyler B. S., Lutz A., Davidson R. J. (2016). Long-term mindfulness training is associated with reliable differences in resting respiration rate. Sci. Rep. 6, 1-6. 10.1038/srep27533].

Heart rate is another measure of autonomic arousal that can be obtained using photoplethysmography embedded in a wearable or directly via the camera of a smartphone. A drop in heart rate may signal the beginning of a period of paying attention to something, and therefore high absorption. [See Kitson, A., Chirico, A., Gaggioli, A., & Riecke, B. E. (2020). A review on re-search and evaluation methods for investigating self-transcendence. Frontiers in psychology, 11.]

Heart rate variability (HRV) has also been used as a measure of transcendence (see Clayton et al., 2019, for example). However, HRV may be less useful than other measures because of individual differences and the requirement of long periods for baseline measurements. [See Clayton R. B., Raney A. A., Oliver M. B., Neumann D., Janicke-Bowles S. H., Dale K. R. (2019). Feeling transcendent? Measuring psychophysiological responses to self-transcendent media content. Media Psychol. 1, 1-26. 10.1080/15213269.2019.1700135.]

Any combination of one or more of the biometric measures described above may be used to generate a state absorption measure 240 in various embodiments. In some examples, the data epochs may be assessed for absorption, and the highest-absorption epochs may then be identified as high-absorption epochs for the purpose of other processes performed by the absorption system 100. For example, epochs of high absorption (e.g., 90th percentile or greater for a given session) may be used to identify flashbulb memory events, as further described below with reference to FIG. 4.

FIG. 4 is a schematic diagram showing identification of flashbulb memory (FBM) events based on the state absorption measure 240. In some embodiments, a FBM event is identified based on whether the state absorption measure 240 is in the highest scale range (e.g., above an absorption threshold, such as a threshold defined by the 90^{th} percentile of epochs within a session) for an epoch of at least 20 seconds (e.g., two consecutive 10-second windows). Identifying an epoch as indicating a FBM event is shown in FIG. 4 as “FBM event =1”; epochs that are not identified as FBM events are indicated as “FBM event =0”. Thus, for example Epoch 1 402 and Epoch 3 406 in FIG. 4 are not FBM epochs, but Epoch 2 404 is a FBM event, based on the epoch-wise state absorption measure 240 generated by the biometric classifier 200.

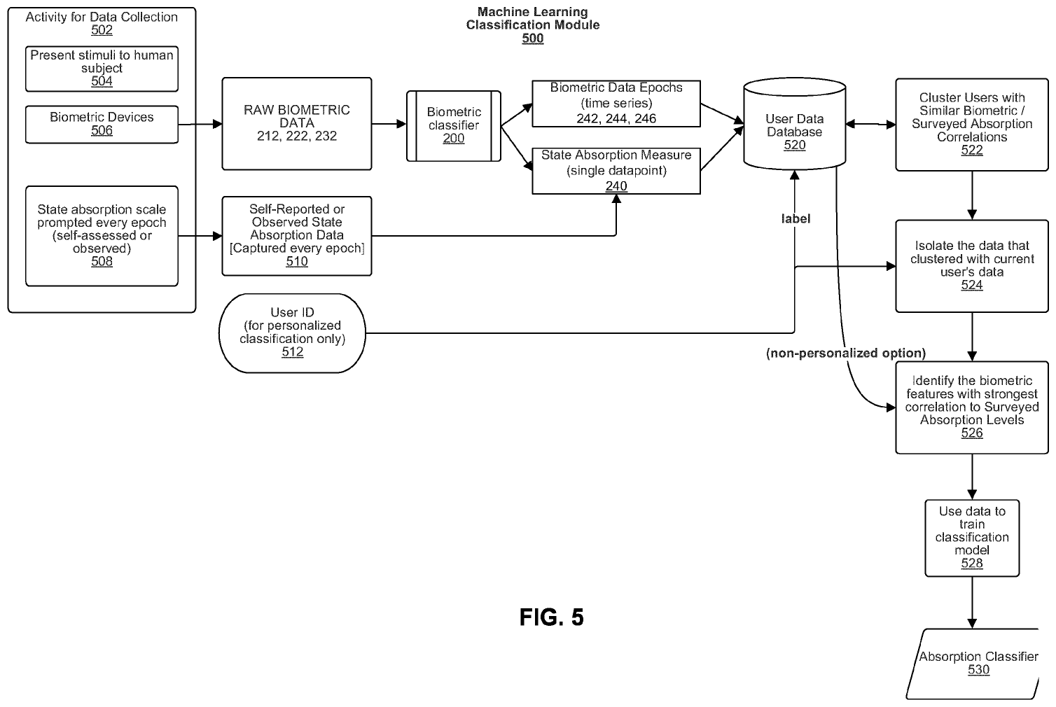

FIG. 5 is a schematic diagram showing a machine learning classification module 500 for state absorption. The biometric classifier 200 may be used to process biometric data (e.g., 212, 222, 232) from one or more biometric devices 506 (such as video cameras, EEG electrode arrays, and fNIRS sensors) while stimuli are being presented 504 to a human subject during data collection 502. The biometric data epochs (e.g., 242, 244, 246) may be output as time series data, along with the state absorption measure 240.

State absorption scale data is also collected in response to prompts (e.g., prompts presented every epoch 508). The state absorption scale data 510 may include self-reported state absorption information reported by the subject and/or state absorption information observed by an observer of the subject (e.g., a therapist). The state absorption measure 240 may be generated at least in part, or may be validated, based on the state absorption scale data 510 in some embodiments.

The biometric data epochs (e.g., 242, 244, 246) and state absorption measure 240 may be stored in a user data database 520 in association with a User ID 512 identifying the subject. At block 522, a clustering process (e.g., using a filtering model or a trained machine learning model) may be applied to the user data database 520 to generate clusters of biometric data epochs (e.g., 242, 244, 246) and state absorption measures 240 for users having similar relationships between the biometric data and the state absorption measure 240. At 524, the user data for the current subject's cluster is isolated. From this isolated set of user data, at 526 the biometric features most strongly correlated with state absorption are identified. At 528, these identified features are used as ground truth information to train a classification model shown as absorption classifier 530 to process the subject's biometric data to generate a predicted state absorption measure 240.

In some embodiments, the absorption classifier 530 is not trained with reference to a specific user, but is instead a non-personalized absorption classifier 530. In such embodiments, steps 522 and 524 may be omitted, and the absorption classifier 530 may be trained using all user data instead of user data from a specific cluster. If data is collected from a diverse population set, a non-personalized general classifier can be trained that prevents the need for a data collection process for every user.

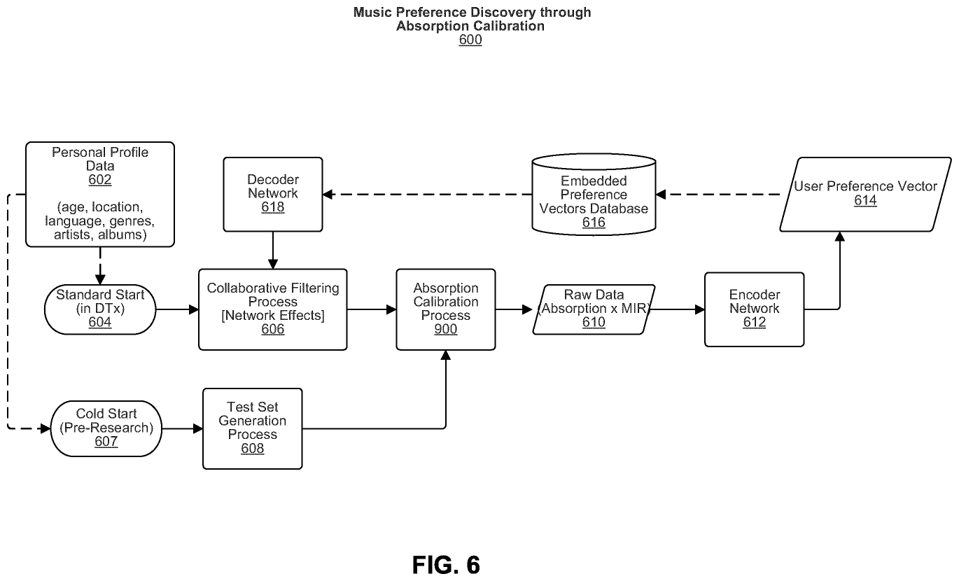

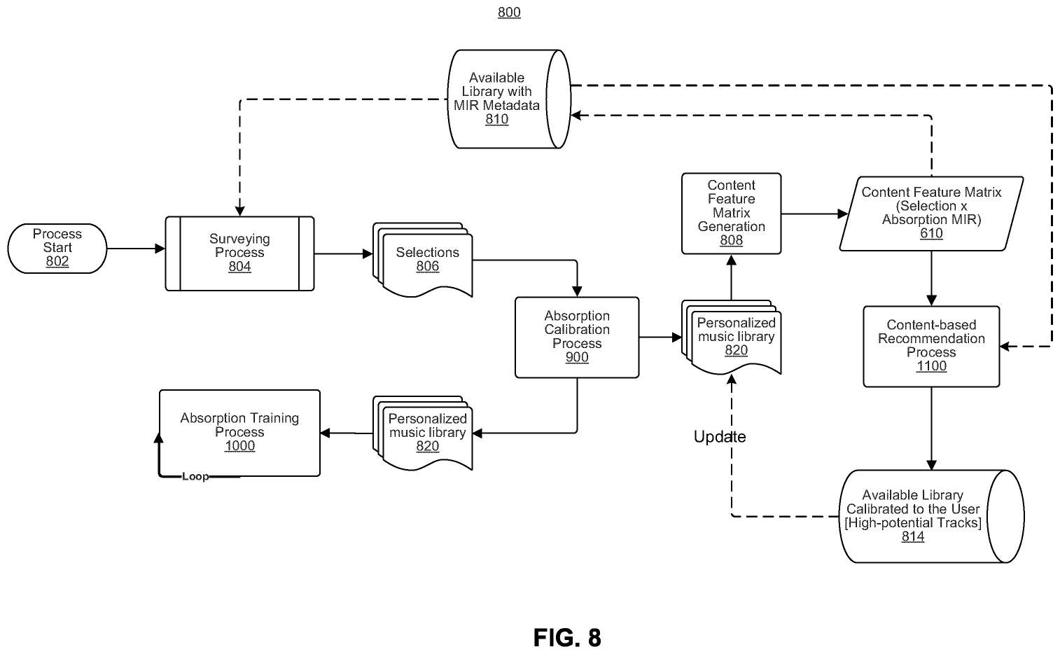

FIG. 6 is a schematic diagram showing a process for music preference discovery through absorption calibration 600. FIG. 8 is a schematic diagram showing generation of a personalized music library 820 based on musical state absorption. FIG. 6 and FIG. 8 show two sets of operations of a common module and will be described jointly.

The process 600 of FIG. 6 begins with an optional phase (beginning at standard start 604) where either a listener or a caregiver (e.g., an observer such as a therapist) is narrowing a large catalogue of music based on characteristic preference assumptions encoded as personal profile data 602 (examples: genres of preference, decade born, geographic locations of significance, lyrical themes of significance). This optional phase would occur when the absorption system 100 affords this type of onboarding and if the music library supplying music segments is tagged with metadata indicating these attributes. This optional phase corresponds to the surveying process 804 of FIG. 8. If this optional phase is followed, selections of music that fit the subject's preferences) will be organized for the next phase in the process; if not, a random selection of testing samples will be selected with a high variance in MIR feature characteristics, providing a wide range of musical aesthetics in order to best validate the breadth of music preferences of the listener. Once these selections are made (shown in FIG. 8 as selection 806), the absorption calibration process 900 can begin. When the standard start 604 process is available, the personal profile data 602 is stored in the database of personal data 619. This stored data in correlation with the data contained in correlation with the vectors stored in Embedded Preference Vectors Database 616. When training the model that is used for the collaborative filtering process 606, the personal profile data 602 stored in the personal profile database 619 is used along with the decoded vectors outputted from the decoder network 218.

The absorption calibration process 900 is designed to assess a listener's music preferences through a listening test measured by traditional state absorption scales and/or classified biometric measures (as described above with reference to FIGS. 2-5). After the absorption calibration process 900, the best (i.e., highest music trait absorption) selections of music segments identified by the absorption calibration process 900 are used to create a matrix 610 of absorption levels and content features (e.g., state absorption measures 240 by MIR features of the music segments), representing the subject's absorption preferences. This matrix 610 is then used in a content-based candidate generation process to create a personalized music library 820 for the subject, calibrated based on absorption levels, as shown in FIG. 8. It is also possible to re-assess samples and continuously update the personalized music library 820 to reflect changes in the listener's state absorption behaviour over time. For example, if it is possible to leverage this test at the beginning of each listening session, this calibration process 900 can be done regularly to best capture the changing preferences of the subject. Updating the subject's preferences can be accomplished by processing the matrix 610 with an encoder network 612 configured to generate a user preference vector 614 encoding the subject's music preferences based on the contents of the matrix 610. The user preference vector 614 may be stored in an embedded preference vectors database 616 along with user preference vectors 614 for other subjects. A decoder network 618 can be used to decode the user preference vectors 614 stored in the embedded preference vectors database 616 to provide the collaborative filtering inputs to the collaborative filtering process 606.

In some examples, the matrix 610 used to generate the user preference vectors 614 is generated using all music segments and corresponding absorption levels, not just the best selections. By including all music segments and corresponding absorption levels in the matrix 610, a more comprehensive and accurate preference vector 614 may be generated. However, when generating the personalized music library 820, some embodiments may generate the matrix 610 using only the best selections.

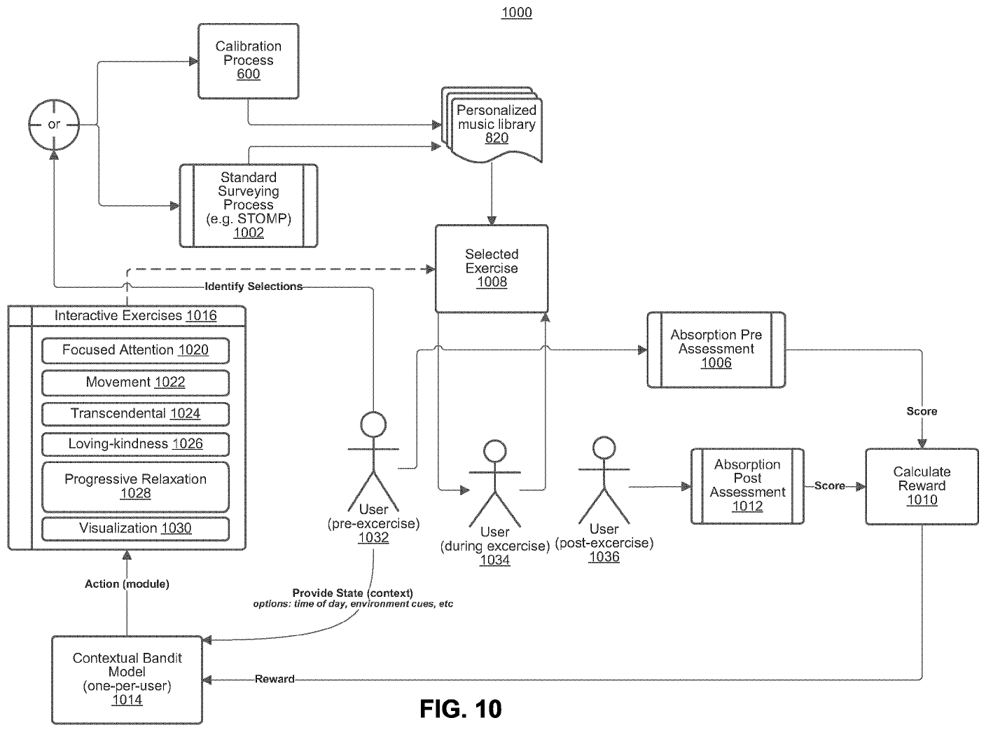

The best selections resulting from the absorption calibration process 900, which are selected to form the personalized music library 820, can also be used for an absorption training process 1000 as shown in FIG. 8 and described in greater detail with reference to FIG. 10. The absorption training process 1000 leverages interactive activities and machine learning optimization to increase the levels of trait absorption in the subject. This absorption training process 1000 can be done multiple times to best increase these trait levels over time. This training would occur before and in between listening sessions for enhanced listening outcomes.

FIG. 8 also shows how the results of the absorption calibration process 900 are used to supplement the personalized music library 820 with music segments other than those directly inducing high state absorption in the subject. During this process, the system leverages objective and/or subjective data capture to assess a listener's music preference/state absorption measures 240 for a variety of music segments played in succession. This data is captured and the selections with the highest absorption levels are separated, and their MIR features (encoded in matric 610 by content feature matrix generation process 808) are analysed or accessed via database 810 (when segments can be analyzed prior to the testing process). A content-based filtering process 1100 using MIR features (e.g., using cosine similarity, Euclidean distance, or unsupervised clustering machine learning) is then leveraged to find other tracks like the segments with high absorption levels (shown as calibrated library 814), and the personalized music library 820 is further populated accordingly.

The personal profile data collected in the surveying process 804 can also be used to reduce the size of the available library for the content-based filtering process 1100, improving the efficiency of that process.

State musical absorption has been empirically determined to be a strong indication of music preference (See Lifshitz (2019)). Critically, this correlation between absorption and preference does not depend on the emotion being conveyed by the music (See Lifshitz (2019)). This may explain why music that conveys negative emotions can be deeply absorbing and enjoyable. It has been suggested that the enjoyment may result from a dissociation between the activated negative emotion and accompanying unpleasant somatic effects (See Van Elk (2019)), which may in turn be underpinned by the tendency of preferred music to activate reward circuitry (See Hu (2017) and [Salimpoor, V. N., van den Bosch, I., Kovacevic, N., McIntosh, A. R., Dagher, A., & Zatorre, R. J. (2013). Interactions between the nucleus accumbens and auditory cortices predict music reward value. Science, 340 (6129), 216-219]). Inducing a negative emotion while activating reward circuitry and without unpleasant somatic effects may increase the likelihood of reframing negative autobiographical memories that may arise in the context of psychedelic-assisted therapy (See Cardona).

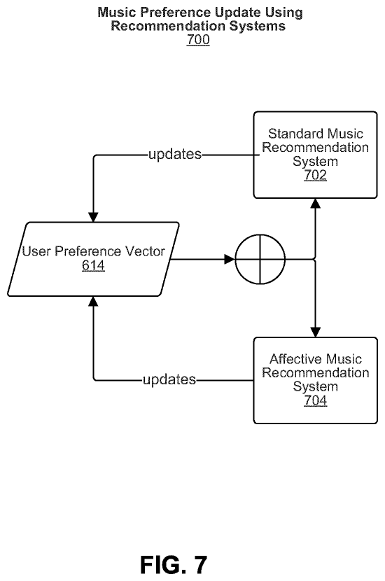

FIG. 7 is a schematic diagram showing a music preference updating process 700 using music recommendation systems. In some embodiments, the subject's music preference data encoded as the user preference vector 614 may be used to inform other music recommendation systems, and to be informed or updated by them in turn. For example, a conventional or standard music recommendation system 702, such as a system generating a preference profile based on listener choices, may use the user preference vector 614 as the baseline user preference profile for the subject. The adjustments to the user preference profile made by the standard music recommendation system 702 may be propagated to the user preference vector 614 as updates. In some examples, an affective music recommendation system 704 may be used similarly to receive the user preference vector 614 as a baseline preference profile and to update it during operation. Affective music recommendations systems 704 may include systems assessing a user's musical preferences based on affective or emotional states induced by different musical stimuli. In some examples, an affective music recommendation system 704 may employ agitation or anxiety as a termination function to cease presenting music deemed to be upsetting to the listener. However, in some embodiments, the state absorption measure 240 computed by the absorption system 100 may be used as the feedback mechanism used to assess anxiety or agitation.

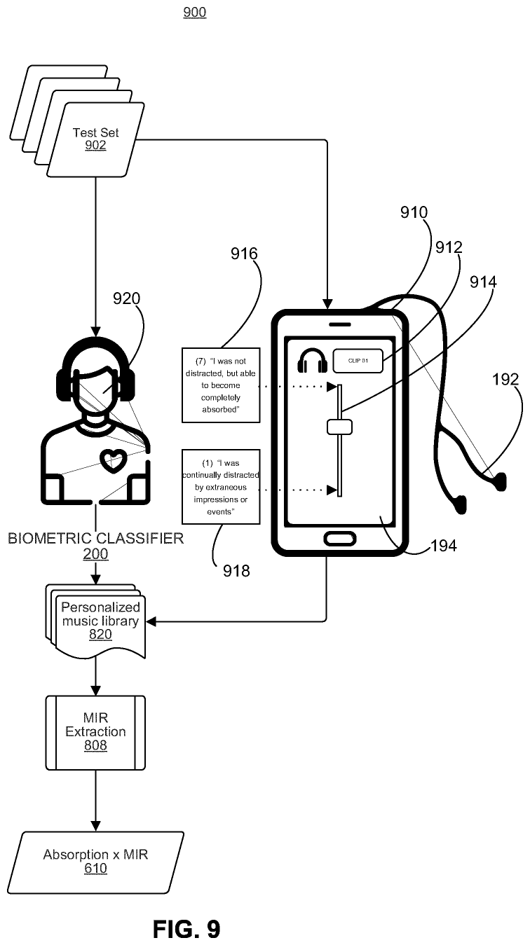

FIG. 9 is a schematic diagram showing an absorption calibration process 900 including a human subject 920 and a user device 910. Traditional music preference surveying relies on either lengthy onboarding processes or implicit data like song “skips” or playback controls (which does not directly indicate the listener's preferences necessarily). The process shown in FIG. 9, in contrast, provides direct insight to the subject's 920 musical preferences and allows for the curation of music that has a likelihood to induce states of enjoyment and transcendence, providing value to therapeutic processes that rely on these states (e.g., psychedelic-assisted therapy) and entertainment use cases (e.g., music streaming platforms).

A music segment (shown as “Clip 1” 912) is presented to the subject 920 via a user device 910 equipped with an audio output 192, such as headphones or speakers. The user can use the touchscreen 194 to select (e.g. using a slider 914) a self-reported level of psychological absorption. A standard absorption scale may be used in some embodiments. In FIG. 9, the slider 914 moves between a maximum absorption position of “(7) I was not distracted, but able to become completely absorbed” 916 and a minimum absorption position of “(1) I was continually distracted by extraneous impressions or events” 918.

In some embodiments, this calibration process 900 allows for a passive calibration of music preferences (via biometrics), which provides a powerful tool to the process of reminiscence therapy when patients are non-communicative and unable to self-assess their own music preferences through traditional surveys and self-assessment modalities. Given that music curation based on preference is a key component to reminiscence therapy, this process could provide a significant advantage to products leveraging music for this form of therapy. The methods described above for measuring naturally occurring absorption markers using biometrics may providing means to objectively determine the music associated with a patient's deepest memories, preventing the need to rely on second-hand accounts. This technology could enable a dramatic leap forward in the standard of care within the reminiscence therapy practice for those living with dementia and brain injury.

FIG. 10 is a schematic diagram showing an absorption training process 1000 according to example embodiments described herein. High levels of musical trait absorption is generally seen as a personality trait, naturally developed through biological predispositions, life experience, musical training, etc. Individuals living with high levels of musical trait absorption find themselves experiencing a connection with music that renders potentially transformative effects on their lives. In therapeutic use cases especially (and even entertainment use cases), trait absorption, or one's “openness to a transformative music experience” may have significant implications on treatment outcomes. Lower levels of trait absorption in some individuals may minimize the efficacy of music-based therapeutic modalities (see Sandstrom & Russo, 2013).

FIG. 10 shows a process 1000 for training individuals to achieve higher levels on the trait absorptions scale, potentially having a massive impact on the subjects' ability to have impactful listening experiences and improved health outcomes post-session. Building on top of existing music trait absorption inventories, the process 1000 provides an interactive audio experience to help subjects increase their trait absorption while leveraging machine learning to optimize the training process for both the individual and the network of listeners. Using the personalized music library 820 resulting from the

absorption calibration process 900 and other processes of FIGS. 6-9, the training process 1000 will select top performing tracks within the library (tracks with the highest ab-sorption levels for the listener) and a variety of interactive exercises (see below) are completed with the intent of enhancing the trait-based absorptive qualities in the listener. These interactive listening activities can feature both full multi-track audio or isolated music stems. Despite the personalized music library 820 (resulting from the calibration process 600) providing a body of content with higher likelihood of success for absorption training, it is possible to perform the training process without the calibration process (e.g., by asking a subject to complete a survey at step 1002 like STOMP, described by [Rentfrow, P. J., & Gosling, S. D. (2003). The do re mi's of everyday life: the structure and personality correlates of music preferences. Journal of personality and social psychology, 84 (6), 1236] and perform trait absorption training on music queried based by those survey results.

The absorption training process 1000 is complemented by a personalized machine learning loop that not only learns which activities and works best with each individual, but learns at a network wide level with models that can receive demographic information and other data to best predict the best absorption training activities for new and returning users. The loop of this system is as follows.

First, music segment selections are identified using the calibration process 600 or through a pre-existing music taste surveying process 1002. The context/state is provided to the contextual bandit model 1014 and the user takes a state-based absorption assessment 1006 (either self-assessed state ab sorption question or via biometric classifier 200).

The contextual bandit model 1014 predicts the training module (i.e., the interactive exercise 1016) with the highest likely reward (as calculated by reward calculation 1010) and the subject completes the selected exercise 1008. Thus, for example, the contextual bandit model 1014 may predict that movement meditation 1022 will result in the highest reward; movement meditation 1022 would then be selected as the selected exercise 1008.

The user takes another state-based absorption assessment 1012 (or alternatively is assessed via the biometric classifier 200) and a reward value is calculated at 1010 based on the success of the selected exercise 1008. This reward is sent to the contextual bandit model 1014 (one model per user) and the model 1014 is trained accordingly.

The interactive exercises 1016 used for musical trait absorption training 1000 have been inspired by the absorption in music scale (AIMS) by Sandstrom & Russo (2013). Items from this scale receiving an item-total correlation of 0.5 or greater were selected and thematically grouped into 6 categories. These categories were subsequently mapped to the closest corresponding mode of meditation (e.g., focused meditation 1020 or loving-kindness meditation 1026). For each category, activities have been developed that are inspired by corresponding questions that enable specific types of music-based meditation. A machine learning model is then used to rotate between and learn the most effective exercises for the user given contextual inputs and a reward, calculated at 1010 based on a trait-based absorption assessment 1012 at the end of each exercise. This is designed to optimize the training process 1000 for the user, improving their trait-based absorption levels quickly and effectively. This machine learning loop can not only optimize this training process 1000, but it can also be used for any similar cognitive training method that has multiple modules as options.

The interactive nature of the absorption system 100 also enables a novel form of meditation. When practiced regularly, these activities are expected to enhance a user's capacity for musical trait absorption. The six types of music-based mediations are as follows: focused meditation 1020, movement meditation 1022, transcendental meditation 1024, loving-kindness meditation 1026, progressive relaxation meditation 1028, and visualization meditation 1030.

The first meditation, focused meditation 1020, is developed in greater detail below in order to provide a concrete example. However, the same general process will be followed for each meditation. The process starts with the selection of music segments that are likely to promote state absorption for a given subject.

Focused meditation 1020 begins with absorption track selections. A learning layer will allow the system to determine what exercises are working optimally for a given subject. Following an initial survey, a new candidate music segment will be played. The subject will be asked to judge it on a 7-point scale that assesses musical state absorption (from Hall et al., 2016), as described above with reference to FIG. 9. This musical state absorption scale will be administered before and after the musical mediation. This sequence (rating-meditation-rating) will be repeated at least three times. Average pre-post changes in state absorption will be used to update the system with regard to the efficacy of that music-based meditation for a given user.

A focused meditation 1020 session begins by asking the subject to block out all distractions by imagining that “nothing exists in the world except for themselves and the music that will follow”. They will next be asked to focus on their breath and to synchronize it with the music that follows. The music will commence with a percussive layer that reinforces the beat and metrical structure of the track (e.g., a metronomic bass drum that has a strong beat at the beginning of each 4-beat cycle). The beat will be further reinforced through the use of a dynamic metronome presented on the user device 910 (e.g., smartphone or tablet).

The instructions to the subject (presented through sound and text) will support synchronization with each metric cycle (e.g., 4-beats). A new layer of the track (e.g., melody harmony) will be added following the completion of the first 8 bars of music. The starter percussion layer will be faded out after 16 bars but the subject will be given instructions to maintain their breath rate with the support of the visual metronome. Once all layers of the track have been added (e.g., after 32 bars), the visual metronome will fade out and the subject will be asked to maintain their breath rate through the completion of the track without the assistance of the visual metronome.

The same basic process of using a learning layer to assess the efficacy of a mediation for a given subject will be used for the 5 other forms of musical meditation. Each of these approaches has been briefly outlined below.

In movement meditation 1022, the subject is instructed to move their hands as though they are conducting the music. The subject will be asked to tap the phone at the rate of the beat, allowing each hit to correspond with the beat onset (this requires anticipation of the beat). The subject will be given a score on their level of sensorimotor synchronization (determined by the mean absolute asynchrony).

In transcendental meditation 1024, the subject is asked to imagine that they are on a small vessel at sea that is being carried by swells; the whole ocean seems to be in upheaval but the depth of the ocean is at rest. The subject is asked to select a mantra and to produce it on the beat (e.g., “hmmm”). Possible instructions while producing the mantra include: allow the music to quiet the mind, allow the music to achieve deep rest.

In loving-kindness meditation 1026, the subject is asked to allow themselves to feel connected to other people through the music, allowing the music to help the subject receive love from others and to send it back. Instruction will ask the subject to empathize with the music and to imagine it as another person, allowing themselves to feel connected to the other person.

In progressive relaxation meditation 1028, the subject will be asked to listen to the music and to slowly tighten and relax different parts of the body (instructions will guide this process). The subject will then be asked to allow the music to guide their awareness of different body parts, progressively relaxing each area.

In visualization meditation 1030, the subject will be asked to imagine that they are listening to the music so vividly that you they are hearing it “live”. The subject will be asked to imagine the colours and images that the music is evoking. The feeling of immersion will be enhanced through the use of audio spacialization methods.

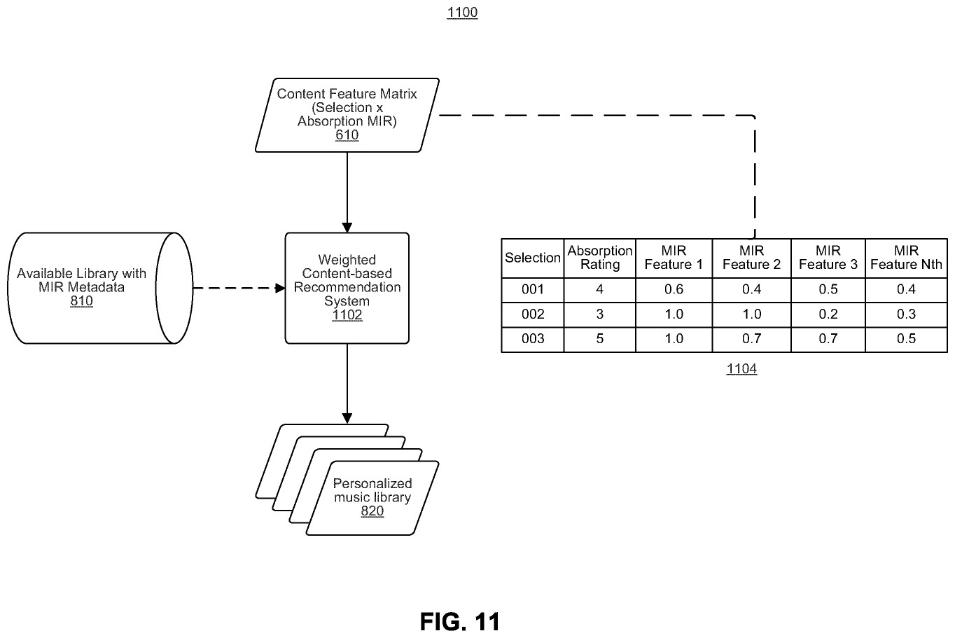

FIG. 11 is a schematic diagram showing a content-based music segment candidate generation process 1100 referred to above in reference to FIG. 8. During the content-based filtering process, a weighted feature-based recommendation system 1102 is used to create a personalized library using a matrix 610 of absorption ratings and MIR features. An example system expands the matrix 610 with other available tracks with their corresponding MIR features (as stored in database 810) and a supervised classification algorithm (e.g., k-nearest neighbour) is used to score the expanded set. Using this system, the resulting personalized music library 820 becomes a selection of music segments in the available library 810 that have a similar aesthetic to the best segments identified during the absorption calibration process. This step provides a scalable means for creating a personalized music library 820 out of a matrix 610 of absorption data correlated with musical features.

Although the present disclosure may be described, at least in part, in terms of methods and devices, a person of ordinary skill in the art will understand that the present disclosure is also directed to the various components for performing at least some of the aspects and features of the described methods, be it by way of hardware components, software or any combination of the two. Accordingly, the technical solution of the present disclosure may be embodied in the form of a software product. A suitable software product may be stored in a pre-recorded storage device or other similar non-volatile or non-transitory computer-or processor-readable medium, including DVDs, CD-ROMs, USB flash disk, a removable hard disk, or other storage media, for example. The software product includes instructions tangibly stored thereon that enable a processing device (e.g., a personal computer, a server, or a network device) to execute examples of the methods or systems disclosed herein.

The skilled person will also appreciate that the output of the methods and devices described above, such as the personalized music library 820, may be stored as music data (such as audio files or playlist data) on a storage medium such as non-volatile or non-transitory computer-or processor-readable medium, including DVDs, CD-ROMs, USB flash disk, a removable hard disk, or other storage media. The music may also be stored on other digital or analog storage media appropriate for use in audio applications or audio playback or broadcast devices, such as cassette tapes, vinyl records, or any other storage medium for digital or analog music data.

In the described methods or block diagrams, the boxes may represent events, steps, functions, processes, modules, messages, and/or state-based operations, etc. While some of the above examples have been described as occurring in a particular order, it will be appreciated by persons skilled in the art that some of the steps or processes may be performed in a different order provided that the result of the changed order of any given step will not prevent or impair the occurrence of subsequent steps. Furthermore, some of the messages or steps described above may be removed or combined in other embodiments, and some of the messages or steps described above may be separated into a number of sub-messages or sub-steps in other embodiments. Even further, some or all of the steps may be repeated, as necessary. Elements described as methods or steps similarly apply to systems or subcomponents, and vice-versa. Reference to such words as “sending” or “receiving” could be interchanged depending on the perspective of the particular device.

The above-described embodiments are considered to be illustrative and not restrictive. Example embodiments described as methods would similarly apply to systems, and vice-versa.

Variations may be made to some example embodiments, which may include combinations and sub-combinations of any of the above. The various embodiments presented above are merely examples and are in no way meant to limit the scope of this disclosure. Variations of the innovations described herein will be apparent to persons of ordinary skill in the art, such variations being within the intended scope of the present disclosure. In particular, features from one or more of the above-described embodiments may be selected to create alternative embodiments comprised of a sub-combination of features which may not be explicitly described above. In addition, features from one or more of the above-described embodiments may be selected and combined to create alternative embodiments comprised of a combination of features which may not be explicitly described above. Features suitable for such combinations and sub-combinations would be readily apparent to persons skilled in the art upon review of the present disclosure as a whole. The subject matter described herein intends to cover and embrace all suitable changes in technology.

1. A method for computing a state absorption measure of a human subject, comprising:
obtaining biometric data from the human subject, the biometric data including one or more of the following:
functional near infrared spectroscopy (fNIRS) data;
electroencephalography (EEG) data;
eye tracking data; and
photoplethysmography (PPG) data; and
processing the biometric data to compute the state absorption measure.

2. The method of claim 1, wherein:
the biometric data includes fNIRS data, and
processing the fNIRS data comprises:
detecting a reduction in default mode network (DMN) activity;
computing the state absorption measure based on the reduction in DMN activity by
computing a ratio of activity between:
the DMN activity; and
a combination of the hMNS activity and the FPN activity; and
computing the state absorption measure based on the ratio of activity;
detecting an increase in human mirror system (hMNS) activity and frontoparietal attention network (FPN) activity; and
computing the state absorption measure based on the increase in hMNS and FPN activity

3-4. (canceled)

5. The method of claim 2, wherein:
the biometric data includes fNIRS data; and
processing the fNIRS data comprises:
removing fNIRS data channels that have saturated or have not received sufficient light, thereby generating pruned fNIRS intensity data;
converting the pruned fNIRS intensity data into fNIRS Optical Density (OD) data;
applying a filter, comprising a low pass or band pass filter, to the INIRS OD data to suppress physiological noise and high frequency instrument noise, thereby generating filtered fNIRS data;
in response to determining that short channels exist, processing the filtered fNIRS data using a general linear model (GLM) to generate GLM fNIRS data;
performing a regression on the GLM fNIRS data to suppress short channel physiological noise, thereby generating regressed fNIRS data;
processing the regressed fNIRS data to generate a plurality of fNIRS epochs;
processing the plurality of fNIRS epochs to detect fNIRS movement within one or more epochs;
in response to detecting movement within one or more epochs, applying movement correction to the one or more epochs to generate a plurality of movement-corrected epochs;
processing the plurality of movement-corrected epochs to generate a plurality of oxygenation (HbO) and deoxygenation (HbR) data epochs based on an optical density of the plurality of movement-corrected epochs;
applying an averaging function to the plurality of oxygenation (HbO) and deoxygenation (HbR) data epochs to generate a plurality of oxygenation epochs, each oxygenation epoch having a respective oxygenation measure;
processing each oxygenation epoch to determine the DMN activity and the hMNS activity for the oxygenation epoch based on the oxygenation measure of the oxygenation epoch; and
computing the state absorption measure for each oxygenation epoch based on the ratio of activity of the DMN activity and the hMNS activity.

6-11. (canceled)

12. The method of claim 2, wherein:
the biometric data includes EEG data; and
processing the EEG data comprises:
detecting a decrease in high-frequency band power of the EEG data, high-frequency band power comprising a power of high-frequency waves comprising alpha waves and beta waves;
detecting an increase in theta wave power of the EEG data; and
computing the state absorption measure by:
computing a ratio between the theta wave power and the high-frequency band power; and
computing the state absorption measure based on the ratio.

13-14. (canceled)

15. The method of claim 12, wherein:
processing the EEG data comprises:
filtering the EEG data to generate filtered EEG data;
subtracting a reference from the filtered EEG data to generate differential EEG data;
processing the differential EEG data to generate a plurality of EEG epochs,
cleaning the plurality of EEG epochs of bad data to generate a plurality of clean EEG epochs; and
for each clean EEG epoch:
computing a normalized theta wave power value based on a power of a 4-7 Hz frequency band of the EEG epoch;
computing a normalized alpha wave power value based on a power of a 8-12 Hz frequency band of the EEG epoch;
computing a normalized beta wave power value based on a power of a 13-20 Hz frequency band of the EEG epoch;
computing the ratio based on the normalized theta wave power value, normalized alpha wave power value, and normalized beta wave power value of the clean EEG epoch; and
computing the state absorption measure of the clean EEG epoch based on the ratio.

16. The method of claim 15, wherein:
filtering the EEG data comprises:
applying a band-pass filter having a high-pass cutoff at 1 Hz and a low-pass cutoff at 55 Hz; and
suppressing powerline interference at 60 Hz;
the plurality of EEG epochs comprise 10-second windows with 5-second overlap between each window and a subsequent window;
the respective powers of the 4-7 Hz frequency band, 8-12 Hz frequency band, and 13-20 Hz frequency band of the EEG epoch are computed by:
determining a power of a 4-20 Hz frequency band of the EEG epoch; and
dividing the power of the 4-20 Hz frequency band between the 4-7 Hz frequency band, 8-12 Hz frequency band, and 13-20 Hz frequency band; and
computing the ratio comprises:
dividing the normalized theta wave power value by the sum of the normalized alpha wave power value and the normalized beta wave power value of the clean EEG epoch;
the method further comprising:
identifying one or more clean EEG epochs having the state absorption measure above a threshold as being high-absorption epochs.

17-23. (canceled)

24. The method of claim 1, wherein:
the biometric data includes eye tracking data;
the eye tracking data comprises video data showing a pupil of an eye of the human subject; and
processing the eye tracking data comprises:
detecting a dilation measure of the pupil; and
computing the state absorption measure based on the dilation measure.

25-26. (canceled)

27. The method of claim 1, wherein:
the biometric data includes PPG data; and
processing the PPG data comprises:
determining a heart rate based on the PPG data;
generating respiration data based on the PPG data; and
computing the state absorption measure based on the heart rate and the respiration data.

28-32. (canceled)

33. The method of claim 1, further comprising:
identifying a flashbulb memory (FBM) potential event based on the state absorption measure by:
detecting a time period during which the state absorption measure is above a FBM threshold for at least a predetermined length of time; and
detecting at least one of creation or recall of a flashbulb memory by the human subject based on the identification of the FBM potential event

34-37. (canceled)

38. The method of claim 1, wherein:
processing the biometric data to compute the state absorption measure comprises:
collecting self-reported state absorption data and training biometric data from one or more training subjects;
training a machine learning model to predict state absorption data for the one or more training subjects based on the training biometric data, using the self-reported state absorption data as semantic labels for supervised learning; and
using the trained machine learning model to generate the state absorption measure for the human subject based on the biometric data of the human subject.

39. (canceled)

40. A method for generating a personalized music library, comprising:
presenting a plurality of music segments to a human subject;
determining a state absorption measure of the human subject during the presentation of each music segment;
computing a musical trait absorption measure for each music segment based on the state absorption measure of the human subject during the presentation of each music segment; and
selecting at least one music segment of the plurality of music segments for inclusion in the personalized music library based on the musical trait absorption measure of the at least one musical segment with respect to the human subject. 41-49. (canceled)

50. The method of claim 40, wherein:
the at least one musical segment is selected for inclusion in the personalized music library based on having a high musical trait absorption measure.

51. The method of claim 50, further comprising:
processing the at least one musical segment selected for inclusion in the personalized music library to generate MIR feature data of the musical segment;
obtaining a plurality of additional music segments, and
selecting one or more of the additional music segments for inclusion in the personalize music library based on a similarity of MIR feature data of the one or more additional music segments to the MIR feature data of the at least one musical segment.

52. The method of claim 51, wherein:
selecting the one or more of the additional music segments for inclusion in the personalize music library based on a similarity of MIR feature data of the one or more additional music segments to the MIR feature data of the at least one musical segment comprises:
obtaining pre-generated MIR feature data of the additional music segments; and
processing the MIR feature data of the at least one musical segment and the pre-generated MIR feature data of the additional music segments to determine their similarity.

53. The method of claim 51, wherein:
selecting the one or more of the additional music segments for inclusion in the personalize music library based on a similarity of MIR feature data of the one or more additional music segments to the MIR feature data of the at least one musical segment comprises:
processing the additional music segments to generate the MIR feature data of the additional music segments; and
processing the MIR feature data of the at least one musical segment and the MIR feature data of the additional music segments to determine their similarity.

54. The method of claim 51, wherein:
the similarity of the MIR feature data of the one or more additional music segments to the MIR feature data of the at least one musical segment is determined using at least one of the following:
cosine similarity;
Euclidian distance; and
a machine learning model trained using unsupervised learning to perform MIR feature clustering.

55-56. (canceled)

57. A method for training a human subject to develop increased trait absorption, comprising:
obtaining a music library comprising a plurality of music segments likely to induce high trait absorption in the human subject;
instructing the human subject to perform one or more interactive exercises; and
while the human subject is performing the interactive exercises, presenting one or more of the plurality of music segments to the human subject.

58-60. (canceled)

61. The method of claim 57, further comprising:
before instructing the human subject to perform the one or more interactive exercises:
presenting a music segment to the human subject; and
determining a first trait absorption measure of the human subject; and
after instructing the human subject to perform the one or more interactive exercises:
presenting another music segment to the human subject; and
determining a second trait absorption measure of the human subject, and
determining an efficacy of the one or more interactive exercises based on a difference between the first trait absorption measure and the second trait absorption measure.

62. The method of claim 57, wherein:
presenting one or more of the plurality of music segments comprises presenting one or more music stems of the one or more of the plurality of music segments.

63. The method of claim 57, wherein:
the one or more interactive exercises includes at least one of the following:
focused meditation;
movement meditation;
transcendental meditation;
loving-kindness meditation;
progressive relaxation meditation; and
visualization meditation.

64-65. (canceled)
